(19) United States
(12) Patent Application Publication
(10) Pub. No.: US 2015/0306182 A1
(43) Pub. Date: Oct. 29, 2015

(54) FAT LOSS COMPOSITION

(71) Applicants: Peter KENNY, (US); Darren DAVEY, (US)

(72) Inventors: Peter KENNY, London (GB); Darren DAVEY, London (GB)

(21) Appl. No.: 14/439,161

(22) PCT Filed: Oct. 29, 2013
(86) PCT No.: PCT/GB2013/052814

§ 371 Date: Apr. 28, 2015

(30) Foreign Application Priority Data
Oct. 29, 2012 (GB) 1219383.5

(51) Int. Cl. A61K 38/27; A61K 31/192

(52) U.S. Cl. CPC A61K 38/27; A61K 31/192

(57) ABSTRACT

Disclosed is a fat loss composition comprising a) a prostaglandin F2alpha analogue, such as cloprostenol, or a pharmaceutically acceptable ester or salt thereof; and b) amino acids 176-191 of human growth hormone. Use of the composition in weight loss, weight management and/or slimming is described.

The present invention relates to a fat loss composition and its use as an aid to weight loss, weight management and/or slimming.

Weight loss, in the context of medicine, health or physical fitness, is a reduction of the total body mass due to a mean loss of fluid, body fat or adipose tissue and/or lean mass, namely bone mineral deposits, muscle, tendon and other connective tissue. It can occur unintentionally due to an underlying disease or can arise from a conscious effort to improve an actual or perceived overweight or obese state.

Intentional weight loss refers to the loss of total body mass in an effort to improve fitness and health, and/or to change appearance. Therapeutic weight loss, in individuals who are overweight or obese, may decrease the likelihood of developing diseases such as diabetes, heart disease, high blood pressure, osteoarthritis, and certain types of cancer.

Weight loss occurs when an individual is in a state of negative thermodynamic flux: when the body is exerting more energy (i.e. in work and metabolism) than it is consuming (i.e. from food or other nutritional supplements), it will use stored reserves from fat or muscle, gradually leading to weight loss.

It is not uncommon for some people who are currently at their ideal body weight to seek additional weight loss in order to improve athletic performance, and/or meet required weight classification for participation in a sport. However, others may be driven by achieving a more attractive body image. Notably, being underweight is associated with health risks such as difficulty fighting off infection, osteoporosis, decreased muscle strength, trouble regulating body temperature and even increased risk of death.

There are many diet plans and recipes that can be helpful for weight loss. Calorie-restriction strategies are one of the most common dietary plans. Notably, daily calorie consumption for dietary purposes varies depending on a number of factors including, age, gender, weight loss goals, and many more. For instance, nutritionists suggest that a minimum of 1,200 calories should be consumed daily by women to maintain health, while the daily calorie consumption by men, on the other hand, could approach 1,500. It is important to note that these recommendations primarily target relatively healthy individuals who seek weight loss for a better body tonus. However, individuals whose obesity places them at an increased risk for diabetes, heart disease, or other conditions, may follow a stricter diet, but only under the close monitoring of a physician and/or specialist. In some cases, obese individuals may need to restrict their daily calorie intake to 800 or even 500. According to the U.S. Food and Drug Administration (FDA), healthy individuals seeking to maintain their weight should consume 2,000 calories per day.

The least intrusive weight loss methods, and those most often recommended, are adjustments to eating patterns and increased physical activity, generally in the form of exercise. Physicians will usually recommend that their overweight patients combine a reduction of processed foods and caloric content of the diet with an increase in physical activity. An increase in fibre intake is also recommended for regulating bowel movements.

Other methods of weight loss include use of drugs and supplements that decrease appetite, block fat absorption, or reduce stomach volume. Application of such medications, however, should only be performed under the strict supervision of a physician and/or specialist.

Surgery (i.e. bariatric surgery) may be used in more severe cases to reduce the size of the stomach artificially, thus limiting the intake of food energy. Bariatric surgery is usually considered a last resort in treating severe obesity and it consists of two main procedures: gastric bypass and gastric banding.

There is a substantial market for products which promise to make weight loss easier, quicker, cheaper, more reliable, or less painful. These include books, DVDs, CDs, creams, lotions, pills, rings and earrings, body wraps, body belts and other materials, not to mention fitness centres, personal coaches, weight loss groups, and food products and supplements. Between $33 billion and $55 billion is spent annually on weight loss products and services, including medical procedures and pharmaceuticals, with weight loss centres garnering between 6% and 12% of total annual expenditure. In Western Europe, sales of weight-loss products, excluding prescription medications, topped £900 million ($1.4 billion) in 2009 (ScienceDaily (Jul. 14, 2010)).

2,4-Dinitrophenol (DNP) was the first synthetic drug that was used for weight reduction in the United States and was used extensively in diet pills from 1933 to 1938. The fat-loss properties of DNP were reportedly first noticed during World War I, when overweight men working with DNP in munitions plants started losing substantial amounts of weight. It did not take very long for this chemical to be identified as the cause. By 1935, probably at least 100,000 people had been treated with DNP in the United States, in addition to many others abroad.

DNP induces weight loss by uncoupling oxidative phosphorylation, thereby markedly increasing metabolic rate and body temperature. While this is an extremely effective way of producing rapid weight loss, there seems to be no ceiling to DNP's temperature increasing effect. Accordingly, DNP overdose will cause fatal hyperthermia. Case reports have shown that an acute administration of 20-50 mg/kg in humans can be lethal. Concerns about dangerous side-effects and rapidly developing cataracts resulted in DNP being discontinued in the United States by the end of 1938. However, DNP continues to be used by some bodybuilders and athletes for rapid loss of body fat. Fatal overdoses are rare, but are still reported on occasion.

Alli™ is an over-the-counter lipase inhibitor produced by GSK Consumer Healthcare to aid weight loss in people who have a body mass index (BMI) of 28 kg/m² or higher. Alli is the reduced-strength version (60 milligrams vs. 120 milligrams) of orlistat (Xenical®), a prescription drug to treat obesity.

Orlistat is not absorbed into the bloodstream, but works locally in the stomach and small intestine where it prevents the action of gastric and pancreatic lipases—the enzymes that break down fats consumed in our diet. Orlistat is taken at mealtimes to prevent fat that is contained in the meal from being broken down and absorbed by the body. This means the body cannot use the fat as a source of energy, or convert it into fat tissue. Instead, the fat is excreted in the faeces.

Orlistat does not cause weight loss on its own but enhances the effects of a low-calorie, low-fat diet and regular exercise and must be taken with a multivitamin to ensure adequate absorption of vitamins A, D, E and K. However, in preventing the absorption of fat from ingested food, orlistat also removes oil from the body, both good and bad oils, and common side effects include oily rectal discharge, passing gas with oily discharge, an urgent need to have a bowel movement, oily or fatty stools, liquid stools, an increased number of bowel movements and a loss of ability to control bowel movements.

Grenade® Fat Burner is a herbal dietary supplement including green tea, bitter orange, caffeine, cayenne, green coffee and phenylalanine. The product acts as an appetite suppressant, a fat burner and increases the body's energy levels, as well as building muscle, to promote weight and fat loss. It should be taken in combination with a high-protein, calorie controlled diet and exercise routine.

However, the most common side effects observed by Grenade users are sudden change of moods, headaches and dizziness, increased rate of heart problems, spasms, experiences of sleeplessness, and irregular bowel conditions. In addition, both green tea and caffeine promote increased anxiety and a major side effect is a feeling of anxiety and nervousness (the jitters). Furthermore, the US National Institute of Health advises against combining bitter orange with caffeine due to an increased risk of cardiovascular complications such as heart failure and hypertension, but both ingredients are present in Grenade.

The present invention seeks to provide an alternative and/or improved weight management product that is at least as effective as products currently available, without the requirement for low or reduced calorie intake and strenuous exercise, or indeed the unpleasant side effects.

Accordingly, from one aspect, the present invention resides in a fat loss composition comprising a) a prostaglandin F2alpha analogue or a pharmaceutically acceptable ester or salt thereof; and b) amino acids 176-191 of human growth hormone, for use in weight loss, weight management and/or slimming.

Preferably, the prostaglandin F2alpha analogue is selected from the group comprising: latanoprost, bimatoprost, fluprostenol and cloprostenol. Most preferably, the prostaglandin F2alpha analogue is cloprostenol or a pharmaceutically acceptable ester or salt thereof, such as cloprostenol sodium.

The chemical name for cloprostenol (also known as oestrophan) is 16-(3-chlorophenoxy)-17,18,19,20-tetranor PGF2alpha (monograph No. 2397 (page 375) of the Merck Index, 11^{th} Edition (1989)) and the IUPAC name is (5Z)-7-[(1R,3R,5S)-2-[(E,3R)-4-(3-chlorophenoxy)-3-hydroxybut-1-enyl]-3,5-dihydroxycyclopentyl]hept-5-enoic acid.

Cloprostenol and its (R)-isomer have powerful luteolytic effects and are primarily used as luteolytic agents causing functional and morphological regression of the corpus luteum, followed by return to oestrus and normal ovulation, in cattle. Cloprostenol is also used for the induction of parturition in pregnant cows, sows and mares.

Cloprostenol is also used by body builders and generally known as PGCL or PG-C1. The compound is used for its weight and water loss: weight loss of 1.5 to 2 pounds (0.68 kg-0.91 kg) per day is normal, while muscle tissue becomes fuller and harder. Food is removed from the gut very quickly, so subjects are able to eat a great deal of protein without feeling full. Most of all, the subjects are able to lose a great deal of body fat without dieting—subjects continue eating their normal diet and lose body fat.

Fat loss and vascularity is markedly improved within days. Reports of dramatic reductions in body fat as measured by calipers, along with enhanced muscle size at the muscles being injected. At the same time, PGCL is believed to act as a diuretic because of the enhanced vascularity and improved physical appearance seen with use. A significant advantage of the compound for body building use is that very little, if any, fat gain is seen in users even when calories are increased substantially.

However, using a minimal dose of 40 μg, within ten minutes users will have to evacuate their bowels. Prolonged diarrhoea for several minutes will be encountered, alongside abdominal pain. Cramping due to dehydration and loss of electrolytes also occurs, while a fall in energy levels, nausea, body temperature and flu-like symptoms are a common occurrence.

The route of administration for body building (injection into muscle mass) requires PGCL to be dosed multiple times during a day. Typically, users inject 20 μg bilaterally (40 μg split between 20 μg on the left and right body parts) three times daily for a total daily dose of 120 μg. Users typically start with a single daily dose of 40 μg and build up to taking it three times a day.

For use in the composition of the invention, a prostaglandin F2alpha analogue dose of between about 10 μg and 100 μg per day, preferably between about 20 μg and 80 μg. Ideally, a dose of about 30 μg per day is optimal for a male and 15 μg per day is believed to be optimal for a female.

The short half life of cloprostenol is a major drawback. Thus, the role of the 176-191 human growth hormone fragment in the composition of the present invention is to enhance and extend the action of cloprostenol.

HGH fragment 176-191(SEQ ID NO: 1: Tyr-Leu-Arg-Ile-Val-Gln-Cys-Arg-Ser-Val-Glu-Gly-Ser-Cys-Gly-Phe) is a stabilised analogue of the growth hormone-releasing factor (GRF) that induces growth hormone (GH) in a specific and physiological manner. To date studies suggest that HGH fragment 176-191 also has several beneficial features for fat reduction in its own right: it reduces abdominal fat (in particular visceral fat) without compromising glycaemia control (blood glucose), increases muscle mass and improves the lipid profile and lipolytic activity.

At a dosage of 500 μg, HGH Fragment 177-191 was shown to increase lipolytic activity in adipose tissue. In other words, this HGH Fragment potently burns body fat, especially stubborn adipose body fat. Of significance is that, in stark contrast to its Human Growth Hormone counterpart, the HGH Fragment has no negative impact on insulin sensitivity, (Ng et al (2000) Horm. Res., 53(6), p. 274-8).

The fragment is similar to Human Growth Hormone, hence the shared amino acid sequence. However, HGH fragment 177-191 does not compete for the hGH receptor and nor does it induce cell proliferation, unlike Human Growth Hormone (Wu and Ng, (1993) Biochem Mol Biol Int., 30(1), p. 187-96). Consequently, this is a very beneficial peptide in terms of burning fat, without unwanted and undesirable side effects. Of particular note is the HGH Fragment's ability to increase IGF-1 levels which translate into the HGH Fragment's ability to give collateral anti-aging and anabolic effects, along with its ability to induce lipolytic (fat burning) activity.

In yet another study, HGH Fragment 177-191 exhibited the ability to burn through adipose tissue by increasing lipolytic activity (the breakdown of fat), in the most stubborn body fat (adipose tissue), while increasing energy expenditure and glucose and fat oxidation in ob/ob mice treated with HGH fragment 177-191. In addition, HGH Fragment 177-191 increased in vitro lipolytic activity and decreased lipogenic activity in isolated adipose tissue from obese rodents and humans (Heffernan et al (2000) Am. J. Physiol. Endocrinol. Metab. 279(3), E501-7).

HGH Fragment 177-191 therefore exhibits the ability to burn through stubborn adipose tissue while increasing energy expenditure, muscle mass, and fat oxidation. All studies have pointed to the fact that the fragment is an effective treatment for obesity and fat loss and safer than its Human Growth Hormone counterpart.

An HGH Fragment 176-191 clinical trial carried out in 2004 (study number AOD9604) indicated that the peptide was highly successful at stimulating the metabolism of body fat in humans and is the first drug to ever do so—all other weight loss drugs (such as Sibutramine) contribute to loss of body weight indirectly by reducing appetite and not actually burning fat itself.

The study was conducted over 3 months at 6 different doses with one of the most interesting conclusions being that the lowest daily dose of 1 mg was found to be the most effective. Since the 1 mg dose was taken orally, it would be the equivalent of approximately 0.25 mg by injection. The average overall weight loss in three months was 3 kg of fat, 300% more than for those test subjects not taking the peptide.

No side effects have been reported with either the clinical trial or from the thousands of people who experiment with HGH Fragment 176-191 for fat loss. Since the peptide has been used in medical circles for many years now, its long-term safety is well established and it can be considered a safe, cheap and effective alternative to full length human growth hormone and other weight loss drugs such as the appetite suppressant, phentermine (phenyl-tertiary-butylamine).

Body builders typically use doses of between 500 μg and 3 mg per day. However, when used in combination with cloprostenol, a dose of between about 200 μg and 1 mg, particularly between 250 μg and 450 μg per day is considered to be suitable. A daily dose equivalent to 2 International Units (approx 660 μg) is considered to be optimal both for men and women.

The components of the composition may be administered sequentially or simultaneously, ideally simultaneously in a single preparation. The prostaglandin F2alpha analogue and human growth hormone fragment may be administered in the same or in separate compositions.

In addition, the combination of components may be administered at any time interval throughout a 24 hour period. For example, dosing in the morning and evening may be found suitable for some users, particularly as the proposed dose of prostaglandin F2alpha analogue is high for a single does. However, the components are taken together once a day, preferably at bed time. Because prostaglandin F2alpha analogues cause a more or less immediate bowel evacuation, taking the composition once a day makes using the fat loss regime of the present invention more comfortable.

Methods for making prostaglandin F2alpha analogues and salts thereof are well known to the skilled person, but additional guidance may be found described in U.S. Pat. No. 5,510,383, U.S. Pat. No. 5,889,052, U.S. Pat. No. 6,723,748, U.S. Pat. No. 5,665,773, U.S. Pat. No. 6,184,250, U.S. Pat. No. 6,344,478, WO 2011/046569 and WO 93/00329.

HGH fragment 176-191 is freely available commercially.

Compositions according to the present invention, and for use in accordance with the present invention, may comprise as, or in addition to the active ingredients, a pharmaceutically acceptable excipient or diluent any suitable binder, lubricant, suspending agent, coating agent, solubilising agent or other materials well known to those skilled in the art. Such materials should be non-toxic and should not interfere with the efficacy of the active ingredient. Acceptable carriers or diluents for therapeutic use are well known in the pharmaceutical art, and are described, for example, in Remington Pharmaceutical Sciences, Mack Publishing Co. (A. R. Gennaro edit. 1985). The precise nature of the carrier or other material will depend on the route of administration, which may be oral, or by injection, e.g. cutaneous, subcutaneous or intravenous.

The composition of the present invention may further comprise additional components such as vitamins, minerals and desirable pharmaceuticals. For example, the addition of Vitamin E imparts anti-oxidative properties to the composition. Essential fatty acids, such as omega 3 and omega 6 may also be included as these promote general health and well being. The composition may further contain a protein source, for example to help boost the immune system or build muscle, or an appetite suppressant.

A variety of methods may be used to deliver the compositions of the present invention. Methods for in vivo delivery include, but are not limited to, bucchal or oral delivery, intravenous delivery, direct or indirect injection (e.g. subcutaneous, intraperitoneal, intramuscular, or other injection methods), topical application, direct exposure in aqueous or media solution, or any of the other commonly used delivery systems known in the art.

Compositions for oral administration may be in tablet, capsule, powder or liquid form. A tablet may comprise a solid carrier such as gelatine or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. Alternatively, the composition may be in a lyophilised form for sublingual administration.

For intravenous, cutaneous or subcutaneous injection, the active ingredients will be in the form of a parenterally acceptable aqueous solution which is pyrogen-free and has suitable pH, tonicity and stability. Those of relevant skill in the art are well able to prepare suitable solutions using, for example, isotonic vehicles such as sodium chloride, Ringer's injection, lactated Ringer's injection, preservatives, stabilisers, buffers, antioxidants and/or other additives may be included, as required. Alternatively, the components may be provided to the user in a dry (e.g. lyophilised) form which is then reconstituted into an injectable solution by the addition of a volume of fluid such as water.

For some applications, pharmaceutical formulation may not be required. For example, the composition of the invention may be tolerated as a pharmaceutical in its own right, without the need for excipients and/or carriers.

Ideally, the composition of the present invention is formulated as a transdermal patch. A transdermal patch is a medicated adhesive patch that is placed on the skin to deliver a specific dose of medication through the skin and into the bloodstream. An advantage of a transdermal drug delivery route over other types of delivery is that a patch provides a controlled release of a composition into the user, usually through either a porous membrane covering a reservoir of medication or through body heat melting thin layers of medication embedded in the adhesive.

The application of the transdermal patch and the flow of active constituents from the patch to the circulatory system via skin occur through various methods.

Iontophoresis passes a few milliamperes of current to a few square centimetres of skin through the electrode placed in contact with the formulation, which facilitates delivery across the skin barrier.

Electroporation is a method of application of short, high-voltage electrical pulses to the skin. After electroporation, the permeability of the skin for diffusion of compounds is increased by four orders of magnitude. The electrical pulses are believed to form transient aqueous pores in the stratum corneum, through which compound transport occurs. It is safe and the electrical pulses can be administered painlessly using closely spaced electrodes to constrain the electric field within the nerve-free stratum corneum.

Application of ultrasound, particularly low frequency ultrasound, has been shown to enhance transdermal transport of compounds, including macromolecules. It is also known as sonophoresis.

Projections called microneedles may also be used to facilitate transdermal compound transport. Needles ranging from approximately 10-100 μm in length are arranged in arrays. When pressed into the skin, the arrays make microscopic punctures that are large enough to deliver macromolecules, but small enough that the user does not feel the penetration or pain. The compound may be coated on the surface of the microneedles to aid in rapid absorption. Alternatively, the microneedles are arrayed on the surface of the patch that comes into contact with the skin. A similar arrangement may be used for oral delivery via sachets that are placed between the cheek and the gum to release the active ingredients.

Use of the composition of the present invention results in the loss of between 0.4 kg and 1 kg per day without the need to cut back on calories or liquid or to incur any strenuous or vigorous workouts.

The prostaglandin F2alpha analogue component is believed to be involved in the breakdown of fat cells. In particular, brown fat cells are believed to be destroyed, absorbed into the body and excreted. This component is also believed to convert white fat cells into brown fat cells which process helps remove excess water from the body. In addition, because brown fat cells are used to generate heat, the body is kept in a thermogenic state, again promoting weight loss. Furthermore, not only does the prostaglandin F2alpha analogue component promote the metabolism of brown fat cells but it also induces fibre type switching in skeletal muscle and multiple aspects of the fasted response seem in the liver. The regulation of these metabolic and cell fate decisions by prostaglandin F2alpha analogue component is achieved through specific interaction with a variety of transcription factors such as nuclear hormone receptors, respiratory factors and muscle specific transcription factors. In this way, not only does this component have an affect on adipocytes (fat tissue), but it also affects cytokines that are secreted by numerous cells, including irisin. Irisin is a hormone researched by scientists at the Harvard Medical School that is believed to replicate some of the positive effects of exercise and increased glucose tolerance to a high fat diet, getting rid of fat instead of storing it.

The growth hormone fragment component of the composition is known to be highly successful in stimulating the metabolism of body fat. This component also promotes repair of skeletal muscle, so muscle wastage is minimised while the fat burning takes place. This is in addition to enhancing the effect of the prostaglandin F2alpha analogue component

The present invention will now be described in more detail with reference to non-limiting examples.

### EXAMPLE 1

Cloprostenol sodium was taken by eight male and two female subjects at a dosage of 120 micrograms for every 2501b (113 kg) body weight. The dosage was calculated pro rata based on the weight of the subject involved.

It was found that giving males shots of 30 μg at a time produced very severe stomach cramps and extensive bowel movements. None of the recipients wished to continue to use this product for more than three days (see Table 1).

With the female subjects the process was identical. However, the dosage was reduced to 15 μg per shot for menstrual reasons. No female subjects wished to continue using this product due to severe stomach cramps.

TABLE 1

| | | | Current | | | | | | Bowel | |
|---|---|---|---|---|---|---|---|---|---|---|
| Test | | Ethnic | Diet (Kcal | Alcohol | Smoker | | Start | Body | Habits | |
| Subject | Sex | Origin | Intake) | (Units) | (Per/Day) | Height | Weight | Fat | (per day) | Comments |
| 1 | M | IC1 | 1800 | 0 | No | 5 ft 11 in | 217 lb | 18% | 6 | Stopped after 3 days |
| | | | | | | (180 cm) | (98 kg) | | | |
| 2 | M | IC4 | 4000 | 0 | No | 5 ft 8 in | 203 lb | 31% | 7 | Stopped after 1 day |
| | | | | | | (173 cm) | (92 kg) | | | |
| 3 | M | IC1 | 2200 | 4 Unit | No | 6 ft | 238 lb | 18% | 6 | Stopped after 2 days |
| | | | | | | (183 cm) | (108 kg) | | | |
| 4 | M | IC1 | 1600 | 0 | No | 5 ft 8 in | 217 lb | 10% | 9 | Stopped after 1 day |
| | | | | | | (173 cm) | (98 kg) | | | |
| 5 | M | IC1 | 2500 | 6 Unit | No | 5 ft 10 in | 231 lb | 15% | 6 | Stopped after 1 day |
| | | | | | | (178 cm) | (105 kg) | | | |
| 6 | F | IC1 | 2000 | 3 Unit | 5 | 5 ft 8 in | 151 lb | 32% | 4 | Stopped as cramp and pain |
| | | | | | | (173 cm) | (68 kg) | | | after first injection |
| 7 | F | IC3 | 2200 | 0 | No | 5 ft 10 in | 173 lb | 30% | 4 | Stopped using as |
| | | | | | | (178 cm) | (79 kg) | | | major discomfort after |
| | | | | | | | | | | 1st injection |
| 8 | M | IC1 | 1300 | 2 Unit | 10 | 5 ft 10 in | 238 lb | 34% | 14 | Stopped after 1st day |
| | | | | | | (178 cm) | (108 kg) | | | due to 14 bowel movements |
| 9 | M | IC4 | 3000 | 0 | No | 5 ft 8 in | 177 lb | 10% | 8 | Stopped after two days as |
| | | | | | | (173 cm) | (80 kg) | | | bowel movements too intense |
| 10 | M | IC1 | 1900 | 3 Unit | No | 5 ft 10 in | 189 lb | 21% | 16 | Stopped on 1st day due |
| | | | | | | (178 cm) | (86 kg) | | | to 16 bowel movements |

For ethnic origin, the IC codes are those used by the British police. IC1 refers to awhile person of northern European type, IC3 refers to an African/Afro-Caribbean persona and IC4 refers to an Indian, Pakistani, Nepalese, Maldivian, Sri Lankan, Bangladeshi, or any other (South) Asian person.

### Example 2

Ten subjects were given a daily subcutaneous dose of 2 International Units (IU) of amino acids 176-191 of human growth hormone for a four week trial period. All subjects completed the four week trial but, as shown in Table 2, there was no real reduction in body fat or meaningful weight loss. All subjects found they felt more energetic and a general good well being.

### Example 3

Eight male subjects were given a subcutaneous dose of 21 U of human growth hormone fragment 176-191 and 30 μg of cloprostenol sodium in one application. Two female subjects were also given a subcutaneous dose of 21 U of human growth hormone fragment 176-191 and 15 μg of cloprostenol sodium per day in one application.

As shown in Table 3, the results show significant weight loss and reduction in body fat with no remarkable side effects in all subjects.

TABLE 2

| | | | Current | | | | | | Bowel |
|---|---|---|---|---|---|---|---|---|---|
| Test | | Ethnic | Diet (Kcal | Alcohol | Smoker | | | | Habits |
| Subject | Sex | Origin | Intake) | (unit) | (Per/Day) | Height | Weight | Body Fat | (per day) |
| 1 | M | IC1 | | 0 | No | 5 ft 11 in | 217 lb | 17% | 1 |
| | | | | | | (180 cm) | (98 kg) | | |
| Week 1 | | | 1800 | | | | | | 1 |
| Week 2 | | | 1800 | | | | | | 1 |
| Week 3 | | | 1800 | | | | | | 1 |
| Week 4 | | | 1800 | | | | 212 lb | 15% | 1 |
| | | | | | | | (96 kg) | | |
| 2 | M | IC4 | | 0 | No | 5 ft 8 in | 203 lb | 31% | 2 |
| | | | | | | (173 cm) | (92 kg) | | |
| Week 1 | | | 4000 | | | | | | 2 |
| Week 2 | | | 4000 | | | | | | 2 |
| Week 3 | | | 4000 | | | | | | 2 |
| Week 4 | | | 4000 | | | | 196 lb | 28% | 2 |
| | | | | | | | (89 kg) | | |
| 3 | M | IC1 | | | No | 6 ft | 238 lb | 18% | 1 |
| | | | | | | (183 cm) | (108 kg) | | |
| Week 1 | | | 2200 | 4 Unit | | | | | 1 |
| Week 2 | | | 2200 | 4 Unit | | | | | 1 |
| Week 3 | | | 3500 | 4 Unit | | | | | 1 |
| Week 4 | | | 2200 | 4 Unit | | | 233 lb | 15% | 1 |
| | | | | | | | (106 kg) | | |
| 4 | M | IC1 | | 0 | No | 5 ft 8 in | 203 lb | 10% | 1 |
| | | | | | | (173 cm) | (92 kg) | | |
| Week 1 | | | 1600 | | | | | | 1 |
| Week 2 | | | 1600 | | | | | | 1 |
| Week 3 | | | 1600 | | | | | | 1 |
| Week 4 | | | 1600 | | | | 203 lb | 10% | 1 |
| | | | | | | | (92 kg) | | |
| 5 | M | IC1 | | | No | 5 ft 10 in | 231 lb | 25% | 1 |
| | | | | | | (178 cm) | (105 kg) | | |
| Week 1 | | | 2500 | 6 Unit | | | | | 1 |
| Week 2 | | | 2500 | 2 Unit | | | | | 1 |
| Week 3 | | | 2500 | 4 Unit | | | | | 1 |
| Week 4 | | | 2500 | 0 | | | 226 lb | 24% | 1 |
| | | | | | | | (103 kg) | | |
| 6 | F | IC1 | | | 5/Day | 5 ft 8 in | 150 lb | 32% | 1 |
| | | | | | | (173 cm) | (68 kg) | | |
| Week 1 | | | 2000 | 3 Unit | 5 | | | | 1 |
| Week 2 | | | 2000 | 2 Unit | 5 | | | | 1 |
| Week 3 | | | 2000 | 1 Unit | 5 | | | | 1 |
| Week 4 | | | 2000 | 4 Unit | 5 | | 147 lb | 30% | 1 |
| | | | | | | | (67 kg) | | |
| 7 | F | IC3 | | 0 | No | 5 ft 10 in | 173 lb | 30% | 1 |
| | | | | | | (178 cm) | (79 kg) | | |
| Week 1 | | | 2200 | | | | | | 1 |
| Week 2 | | | 2200 | | | | | | 1 |
| Week 3 | | | 2200 | | | | | | 1 |
| Week 4 | | | 2200 | | | | 168 lb | 28% | 1 |
| | | | | | | | (76 kg) | | |
| 8 | M | IC1 | | | 10/Day | 5 ft 10 in | 238 lb | 34% | 1 |
| | | | | | | (178 cm) | (108 kg) | | |
| Week 1 | | | 1300 | 2 Unit | 10 | | | | 1 |
| Week 2 | | | 1300 | 2 Unit | 10 | | | | 1 |
| Week 3 | | | 1300 | 2 Unit | 10 | | | | 1 |
| Week 4 | | | 1300 | 2 Unit | 10 | | 226 lb | 30% | 1 |
| | | | | | | | (103 kg) | | |
| 9 | M | IC4 | | 0 | No | 5 ft 8 in | 175 lb | 10% | 1 |
| | | | | | | (173 cm) | (79 kg) | | |
| Week 1 | | | 3000 | | | | | | 1 |
| Week 2 | | | 2900 | | | | | | 1 |
| Week 3 | | | 2800 | | | | | | 1 |
| Week 4 | | | 2600 | | | | 175 lb | 10% | 1 |
| | | | | | | | (79 kg) | | |
| 10 | M | IC1 | | | No | 5 ft 10 in | 189 lb | 21% | 2 |
| | | | | | | (178 cm) | (86 kg) | | |
| Week 1 | | | 1900 | 3 Unit | | | | | 2 |
| Week 2 | | | 1900 | 3 Unit | | | | | 2 |
| Week 3 | | | 1900 | 3 Unit | | | | | 2 |
| Week 4 | | | 1900 | 3 Unit | | | 182 lb | 18% | 2 |
| | | | | | | | (83 kg) | | |

1. A fat loss composition comprising:
a) a prostaglandin F2alpha analogue or a pharmaceutically acceptable ester or salt thereof; and
b) amino acids 176-191 of human growth hormone,
for use in weight loss, weight management and/or slimming.

2. A fat loss composition according to claim 1, wherein the prostaglandin F2alpha analogue is selected from the group comprising: latanoprost, bimatoprost, fluprostenol and cloprostenol.

3. A fat loss composition according to claim 1, wherein the prostaglandin F2alpha analogue is cloprostenol sodium.

4. A fat loss composition according to claim 1, wherein the prostaglandin F2alpha analogue is present in a concentration of between about 5 μg and 60 μg per day.

5. A fat loss composition according to claim 1, wherein the prostaglandin F2alpha analogue is present in a concentration of about 15 μg or about 30 μg.

6. A fat loss composition according to claim 1, wherein the HGH fragment 176-191 is present in a concentration of between about 300 μg and 1 mg.

7. A fat loss composition according to claim 1, wherein the HGH fragment 176-191 is present in a concentration of about 660 μg (2 International Units).

8. A fat loss composition according to claim 1, wherein the composition is administered once a day.

9. A fat loss composition according to claim 1, wherein the components of the composition are administered simultaneously.

10. A fat loss composition according to claim 1, wherein the composition further includes any one or all of: a vitamin, a mineral, a protein source and/or and a physiologically acceptable carrier or excipient.

11. A fat loss composition according to claim 1, wherein the composition is formulated for topical or sublingual delivery.


SEQUENCE LISTING (1 sequence)

SEQ ID NO: 1 - protein, 16 aa, Human
YLRIVQCRSVEGSCGF